(19) United States
(12) Patent Application Publication
(10) Pub. No.: US 2025/0261864 A1
(43) Pub. Date: Aug. 21, 2025

(54) Ambulatory Breath Analyzer and Uses Thereof

(71) Applicants: Joshua Hale, College Station, TX (US); Carl Tong, College Station, TX (US); Alexandre Kolomenski, Houston, TX (US); Hans Schuessler, College Station, TX (US)

(72) Inventors: Joshua Hale, College Station, TX (US); Carl Tong, College Station, TX (US); Alexandre Kolomenski, Houston, TX (US); Hans Schuessler, College Station, TX (US)

(73) Assignee: The Texas A&M University System, College Station, TX (US)

(21) Appl. No.: 19/057,289

(22) Filed: Feb. 19, 2025

Related U.S. Application Data
(60) Provisional application No. 63/555,154, filed on Feb. 19, 2024.

(51) Int. Cl. A61B 5/0205; A61B 5/00; A61B 5/029; A61B 5/083; A61B 5/087; A61B 5/097

(52) U.S. Cl. CPC A61B 5/0205; A61B 5/0836; A61B 5/087; A61B 5/097; A61B 5/7278; A61B 5/742; A61B 5/029; A61B 2560/0431; A61B 2560/0443

(57) ABSTRACT

Provided herein is a portable, handheld, non-invasive device for measuring and monitoring cardiac output in a subject. The device has a mouthpiece with a breath particle filter at the inlet on the device such that exhaled breath flows through the mouthpiece-filter into a chamber containing a CO₂ meter and flow meter to measure CO₂ concentration and volumetric breath flow. Measurements are sent as input data to a computer monitored by an operator whereupon an algorithm calculates cardiac output which is displayed or stored.

### CROSS-REFERENCE TO RELATED APPLICATIONS

This non-provisional patent application claims benefit of priority under 35 U.S.C. § 119 (e) of provisional patent application U.S. Ser. No. 63/555,154, filed Feb. 19, 2024, the entirety of which is hereby incorporated by reference.

### BACKGROUND OF THE INVENTION

### Field of the Invention

The present invention relates generally to the fields of cardiac care and cardiac output measurement devices. More specifically, the present invention relates to a handheld, non-invasive medical device to measure carbon dioxide and flow of exhaled breath to determine cardiac output in outpatient and at home settings.

### Description of the Related Art

A failing heart produces low cardiac output (CO) that causes multi-organs damage and symptoms. Heart failure (HF) treatments aim to restore heart function to normalize cardiac output. Thus, knowing patients' cardiac output at home and at a clinic can optimize treatment and detect deterioration to trigger timely intervention. Current methods involve the use of a heart catheter and probes placed in large arteries. These methods are very invasive, and are typically only used for patients hospitalized for very severe heart problems. Due to their invasive nature and sensitive methods, current measurements of cardiac output need to be performed in specifically equipped hospitals using bulky equipment and require expert technicians, reading physicians, and insurance pre-approval.

Cardiac output can be measured from exhaled breath. The heart delivers blood with high CO₂ to the lungs. The lungs then exchange CO₂ in the blood for oxygen, and exhalation expels the CO₂ from the lungs. Thus, cardiac output determines the amount of expelled CO₂. Then, cardiac output can then be determined by analyzing the CO₂ content of the exhaled breath. The currently accepted method for performing these measurements is the ‘inert gas rebreathing technique’. This method introduces inert blood-soluble gas to the inhaled air of the patient. The rate of change of inert gas concentration in exhaled breath is directly related to the cardiac output of the patient. Cardiac output rebreathing systems are normally used on intubated patients, and require matching of breath flow tubing to the patient lung anatomy. Rebreathing systems cannot be used continuously without risk of patient suffocation and require multiple breaths per measurement.

The high levels of humidity in exhaled breath can negatively impact measurements. Current methods for handling humidity in breath measurements include filtering, sampling, and trapping water in the breath flow. By design, all of these methods restrict the flow of exhaled breath and redirect it over large surface areas in order to reduce condensation. This negatively impacts measurements by smoothing out readings, reducing the effective response time of the sensor. Since CO₂ levels vary rapidly over time during exhalation, these effects can be seriously detrimental to the quality of readings.

Thus, there remain unmet needs in the art for portable, non-invasive medical devices to measure cardiac output without utilizing rebreathing techniques that are usable in a variety of environments. More particularly, the art is deficient in ambulatory breath analyzers for measuring in real-time cardiac output resulting from each breath exhaled into the analyzer without rebreathing. The present invention fulfills this longstanding need and desire in the art.

### SUMMARY OF THE INVENTION

The present invention is directed to a device for analyzing breath exhalations in a subject. The device comprises a housing, a sensor rig disposed within the housing; and a computer operably connected to the sensor rig and comprising an algorithm configured to collect and process data and to display results. The present invention is directed to a related device where the housing further comprises a second filter in fluid connection with an outlet from the sensor rig.

The present invention is further directed to a method for measuring cardiac output in a subject. In this method, a breath is exhaled by the subject through a mouthpiece and first filter combination into the sensor rig in the device described herein. A concentration of carbon dioxide in the breath and volume of breath exhaled by the subject are measured via a CO₂ meter and a flow meter disposed within a flow chamber within the sensor rig. The present invention is directed to a related method further comprising repeating the method steps at least once to monitor levels of cardiac output in the subject. The present invention is directed to another related method further comprising heating the flow chamber to prevent formation of condensation from the breath exhaled therein.

The present invention is directed further to a tool to monitor cardiac output in real-time in a patient in need thereof. The device has a portable, handheld breath analyzer that comprises a housing with a hand grip attached to an exterior bottom surface thereof and a mouthpiece and particle filter subassembly attached in fluid connection to an exterior front surface thereof. A flow chamber-CO₂ meter subassembly is disposed within the housing and is in fluid connection with the particle filter and comprises opposing baffles disposed within the flow chamber and a resistive heater band disposed on an exterior surface thereof where the CO₂ meter is inserted into the flow chamber to contact the opposing baffles. A flow meter is disposed in fluid connection with the CO₂ meter and a computer is in electronic connection with the CO₂ meter and the flow meter, where the computer is configured to process measurements received as input data therefrom via an algorithm. A related tool further comprises another particle filter disposed on a back surface of the housing and in fluid connection with the flow meter.

The present invention is directed further still to a method for monitoring cardiac output in real time in a patient in need thereof. In this method, the breath analyzer described herein is handheld by the patient and a breath is exhaled into the mouthpiece and particle filter subassembly, where the breath flows into the flow chamber-CO₂ meter subassembly. A carbon dioxide concentration in the breath is measured as it flows through the CO₂ meter and the volume of the breath is measured as it flows into the flow meter. The measured carbon dioxide concentration and the volume of the exhaled breath are input into the algorithm and the measured carbon dioxide concentration and volume of exhaled breath are processed as cardiac output data. The cardiac output data is output to a display. The method steps are repeated at least once over a period of time, thereby monitoring the cardiac output of the patient.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIGS. 1A-1D are perspective views of the assembled breath analyzer.

FIG. 2 illustrates the components of the breath analyzer.

FIGS. 3A-3C illustrate the assembly of the mouthpiece-first filter sub-assembly (FIGS. 3A-3B) and of securing the optional second filter to the flow meter (FIG. 3C).

FIG. 4 illustrates the assembly of the resistive heater sub-assembly.

FIGS. 5A-5C illustrates the assembly of the flow chamber (FC)-CO₂ meter subassembly (FIGS. 5B-5C) and the orientation of the CO₂ meter to the baffles within the flow chamber (FIG. 5C).

FIG. 6 illustrates the placement and securement of the flow meter within the case base.

FIG. 7 illustrates how the hand grip is secured to the case base.

FIG. 8 shows an example of the universal interface during measurement of cardiac output.

FIG. 9 is a flowchart of the algorithm used in the breath analyzer to determine cardiac output.

FIG. 10 is a plot of the regression line derived from equation 6 compared to known partial pressures of CO₂ in mixed venous and arterial blood obtained over the course of exercise (3). Polynomial fit is degree 3 where R\hat{ }2=0.9959.

FIG. 11 compares cardiac output comparisons with echocardiogram measurements.

FIG. 12 compares difference vs. cardiac output average in a Bland-Altman analysis of cardiac output.

FIG. 13 compares Cardiac Index (CI) measurements from echocardiogram measurements and from breath. r=0.8239, R\hat{ }2=0.6787, and p<0.0001.

FIG. 14 compares difference vs. average in a Bland-Altman analysis of cardiac index.

FIGS. 15A-15C shows breath analyzer measurements (n=10) post exercise over time for flow rate (FIG. 15A), CO2 concentration percent (FIG. 15B) and cardiac output (FIG. 15C).

FIGS. 16A-16C shows the results of calibration testing of the breath analyzer calculated cardiac output vs. an echocardiogram reference. FIG. 16A is a linear transform when the breath analyzer is uncalibrated (FIG. 16A). FIG. 16B is a two-point calibration per subject for all breaths of the breath analyzer. FIG. 16C is a one-point calibration per subject for all breaths of the breath analyzer.

### DETAILED DESCRIPTION OF THE INVENTION

As used herein, the articles “a” and “an” when used in conjunction with the term “comprising” in the claims and/or the specification, may refer to “one”, but it is also consistent with the meaning of “one or more”, “at least one”, and “one or more than one”. Some embodiments of the invention may consist of or consist essentially of one or more elements, components, method steps, and/or methods of the invention. It is contemplated that any composition, component or method described herein can be implemented with respect to any other composition, component or method described herein.

As used herein, the term “or” in the claims refers to “and/or” unless explicitly indicated to refer to alternatives only or the alternatives are mutually exclusive, although the disclosure supports a definition that refers to only alternatives and “and/or”.

As used herein “another” or “other” may mean at least a second or more of the same or different claim element or components thereof.

As used herein, the terms “comprise” and “comprising” are used in the inclusive, open sense, meaning that additional elements may be included.

As used herein, the terms “consist of” and “consisting of” are used in the exclusive, closed sense, meaning that additional elements may not be included.

As used herein, the term “about” refers to a numeric value, including, for example, whole numbers, fractions, and percentages, whether or not explicitly indicated. The term “about” generally refers to a range of numerical values (e.g., ±5-10% of the recited value) that one of ordinary skill in the art would consider equivalent to the recited value (e.g., having the same function or result). In some instances, the term “about” may include numerical values that are rounded to the nearest significant figure.

As used herein, the ordinal adjectives “first” and “second”, unless otherwise specified, are used to describe a common object and merely indicate that different instances of like objects are being referred to, and are not intended to imply that the objects so described must be in a given sequence, either temporally, spatially, in ranking, or in any other manner.

As used herein, the terms “ambulatory breath analyzer for measuring cardiac output” or “ABAMCO” and “breath analyzer” are used interchangeably and refer to the device presented herein.

As used herein, the terms “subject” and “patient” are used interchangeably and refer to an adult human of any age.

In one embodiment of the present invention, there is provided a device for analyzing breath exhalations in a subject, comprising a housing; a sensor rig disposed within the housing; and a computer operably connected to the sensor rig and comprising an algorithm configured to collect and process data and to display results.

In an aspect of this embodiment, the housing may comprise a hand grip attached to a bottom surface of the housing; and a mouthpiece and a first filter combination in fluid connection, where the first filter is in fluid connection with an inlet to the sensor rig. Further to this aspect, the housing may comprise a second filter in fluid connection with an outlet from the sensor rig. In both aspects of this embodiment the mouthpiece may be disposable.

In another aspect of this embodiment, the sensor rig may comprise a flow chamber in fluid communication with the mouthpiece and a first filter combination; a CO₂ meter inserted within the flow chamber and in fluid communication therewith; and a flow meter attached to the flow chamber in fluid communication with the CO₂ meter and configured to measure flow rate of breath through the flow chamber; where the flow meter comprises a display configured to receive processed data. In this aspect, the flow chamber may comprise a heated band disposed exteriorly on a surface of the flow chamber; and a pair of baffles disposed within the flow chamber. Particularly in this aspect, the CO₂ meter is inserted upside down into and perpendicular to the flow chamber to rest on the set of baffles. In this aspect the CO₂ meter may comprise non-dispersive infrared sensors.

In another embodiment of this invention, there is provided a method for measuring cardiac output in a subject, comprising exhaling a breath by the subject through a mouthpiece and first filter combination into the sensor rig in the device as described supra; and measuring a concentration of carbon dioxide in the breath and volume of breath exhaled by the subject via a CO₂ meter and a flow meter disposed within a flow chamber within the sensor rig. Further to this embodiment, the method comprises repeating the method steps at least once to monitor levels of cardiac output in the subject. In both embodiments, the subject may be a healthy subject or a patient with a cardiac dysfunction.

In an aspect of both embodiments, the measuring step may comprise receiving the breath into the CO₂ meter disposed within a flow chamber in the sensor rig; measuring a carbon dioxide concentration in the breath via the CO₂ meter; measuring the volume of the breath via the flow meter in fluid communication with the flow chamber; inputting the measured carbon dioxide concentration and the volume of the exhaled breath into the algorithm; processing the measured carbon dioxide concentration and volume of exhaled breath as cardiac output data; and outputting the cardiac output data to a display. Further to this aspect, the method comprises heating the flow chamber to prevent formation of condensation from the breath exhaled therein.

In another aspect of both embodiments, the receiving step may comprise holding the device via a grip secured thereto; self-inserting the mouthpiece into the mouth of the subject; and exhaling the breath into the mouthpiece and first filter combination thereby flowing into the flow chamber.

In yet another embodiment of the present invention, there is provided a tool to monitor cardiac output in real-time in a patient in need thereof, comprising a portable, handheld breath analyzer, comprising a housing with a hand grip attached to an exterior bottom surface thereof and a mouthpiece and particle filter subassembly attached in fluid connection to an exterior front surface thereof; a flow chamber-CO₂ meter subassembly disposed within the housing and in fluid connection with the particle filter and comprising opposing baffles disposed within the flow chamber and a resistive heater band disposed on an exterior surface thereof, said CO₂ meter inserted into the flow chamber to contact the opposing baffles; a flow meter disposed in fluid connection with the CO₂ meter; and a computer in electronic connection with the CO₂ meter and the flow meter, where the computer is configured to process measurements received as input data therefrom via an algorithm.

Further to this embodiment, the tool comprises another particle filter disposed on a back surface of the housing and in fluid connection with the flow meter. In both embodiments, the CO₂ meter may be inserted upside down into and perpendicular to the flow chamber. Also, in both embodiments, the CO₂ meter may comprise non-dispersive infrared sensors.

In yet another embodiment of the present invention, there is provided a method for monitoring cardiac output in real time in a patient in need thereof, comprising handholding the breath analyzer as described supra by the patient and exhaling a breath into the mouthpiece and particle filter subassembly, said breath flowing into the flow chamber-CO₂ meter subassembly; measuring a carbon dioxide concentration in the breath as it flows through the CO₂ meter; measuring the volume of the breath as it flows into the flow meter; inputting the measured carbon dioxide concentration and the volume of the exhaled breath into the algorithm; processing the measured carbon dioxide concentration and volume of exhaled breath as cardiac output data; outputting the cardiac output data to a display; and repeating the method steps at least once over a period of time, thereby monitoring the cardiac output of the patient. In this embodiment, the patient may have a cardiac dysfunction.

Provided herein is a device, i.e., an Ambulatory Breath Analyzer for Measuring Cardiac Output (ABAMCO), utilized to determine cardiac output from carbon dioxide (CO₂) and volume of exhaled breath in a non-invasive manner. The subject or patient exhales into a portable handheld device. The device has a unique combination of an air mixing baffle, optical CO₂ quantification, pathway heating to prevent condensation, flow rate metering, and an algorithm to measure cardiac output in real time via a novel CO₂-Modified Fick equation. This device solves problems of condensing moisture, the need for measuring multiple gases, the need for measuring both inhalation/exhalation gases, rebreathing induced false dilutions, and immobile instrumentation.

Particularly, the device measures cardiac output, and the changes in cardiac output over time. The use of breath in order to analyze cardiac output enables collection of heart data in a manner that is simple, comfortable, and harmless to the patient. This information may be read immediately by an operator to make use of the valuable heart metrics.

Moreover, the device is portable and handheld, except for the operating computer, and when using a laptop may be operated exclusively on a dc battery supply. The light weight, small size, and flexibility in power source allow for extreme flexibility in the use environment. Further, the elimination of bulky humidity compensation techniques in favor of the special resistive heater makes the system smaller and even more lightweight.

Furthermore, a CO₂ meter containing at least one CO₂ sensor is mounted in a flow chamber with a novel resistive heater band thereon. This enables collection of CO₂ data directly from the main flow of breath while eliminating the need for extra humidity compensation methods. The resistive heater uses heat to mitigate the negative effects of condensation without the detrimental effects of measures such as filtering or pump sampling of the breath would introduce to the sensor readings. This enables accurate real-time measurement of the CO₂ concentration of breath to produce good quality cardiac output measurements.

Also provided are methods for measuring and monitoring cardiac output in a subject. The breath analyzer may be used in a clinic or at home by healthy subjects or patients with, for example, heart failure or other cardiovascular conditions or cardiac dysfunction. In non-limiting examples, the breath analyzer is a tool to detect and diagnose cardiac dysfunction, to guide heart failure treatment, and guide enhancement of sport or athletic performance. One of ordinary skill in the, for example, a cardiologist or sports medicine physician, is well-able to determine a usage regimen for the subject or patient depending on the age, health and/or prognosis thereof and whether or not measurements should be taken in a clinic setting or taken in-home.

The present embodiments are best described by reference to those figures illustrating the same, but are not meant to limit the present invention in any fashion.

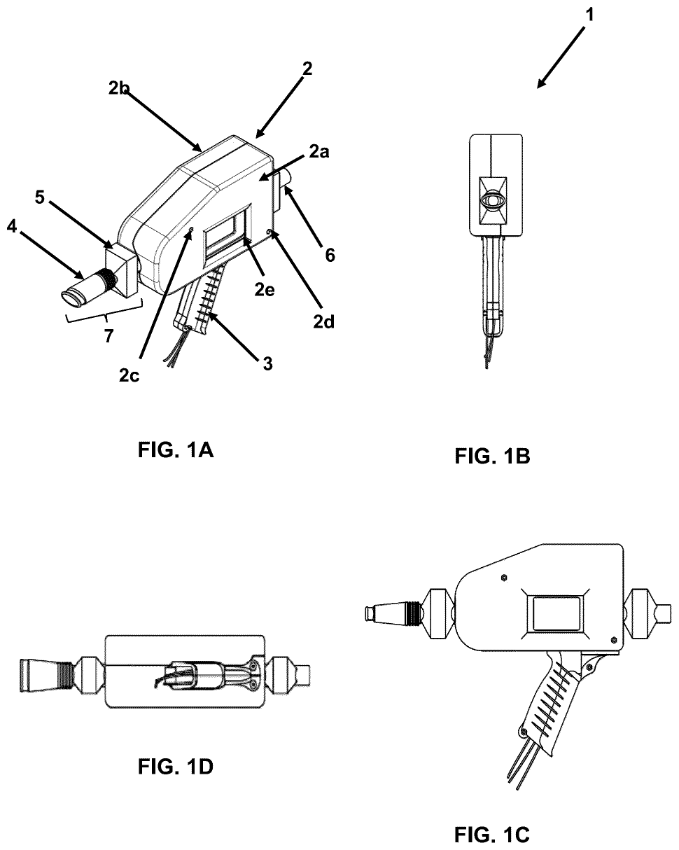

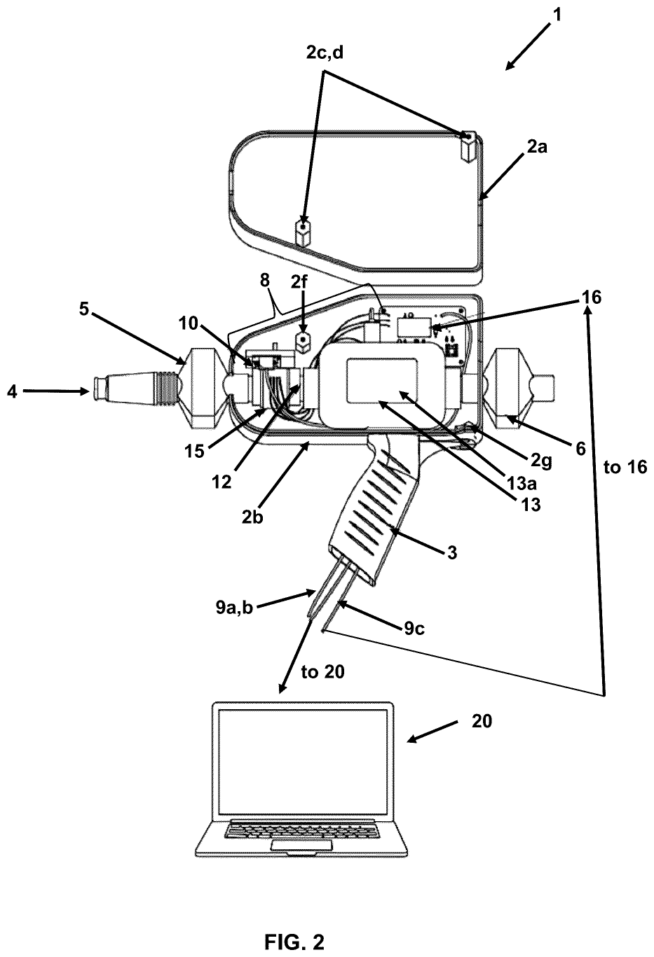

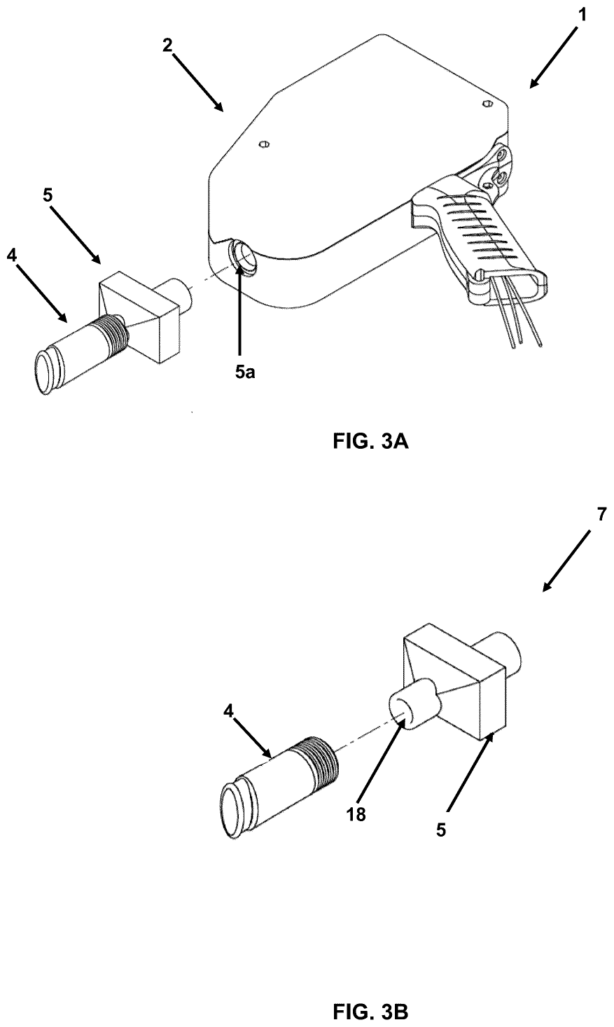

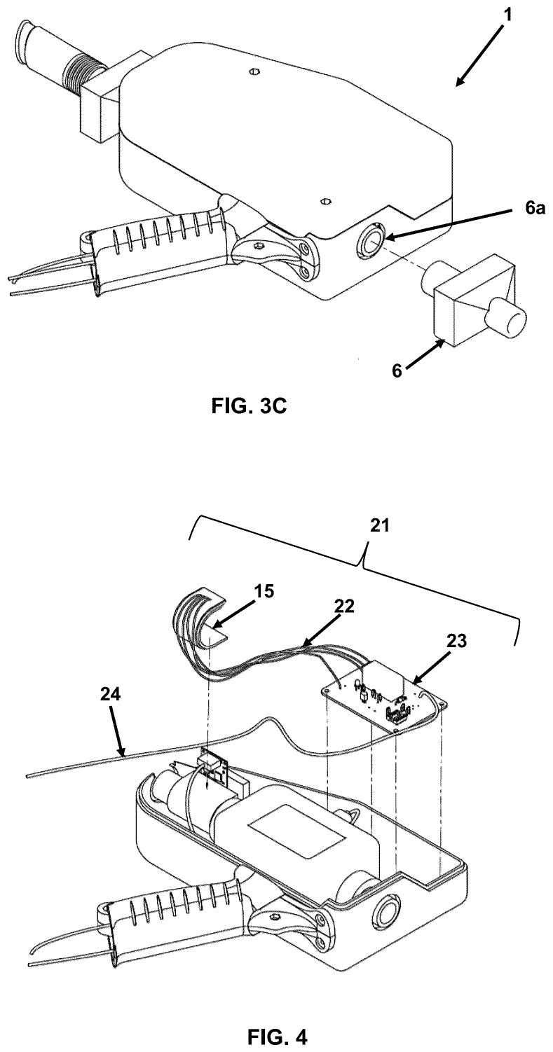

FIGS. 1A-1D show respective a perspective front, top and right side view, a front view, a right side view, and a bottom view of the fully assembled breath analyzer 1. The perspective view of FIG. 1A shows the housing 2 which is formed from the case lid 2a and the case base 2b which are secured together via 3 mm screw holes 2c,d on the case lid. The case lid has an opening 2e into the interior of the housing through which a display may be viewed (see FIG. 2). A hand grip or case handle 3 is attached to the bottom surface of the case base. A mouthpiece 4 is connected to a first filter 5 in combination or as a sub-assembly 7 which is attached at 5a to the front surface of the housing and in fluid connection with the interior of the housing (see FIG. 3A). A second filter 6 is attached at 6a to the back surface of the housing and in fluid connection with the interior of the housing (see FIG. 3C).

With continued reference to FIG. 1A, FIG. 2 shows the exterior and interior components of the breath analyzer 1. The interior of the case lid 2a or top portion of the housing 2 shows the position of the screw holes 2c,d through which the screws (not shown) threadably engage the corresponding screw-type mounting holes 2f,g shown on the interior of the case base 2b. The case base also has screw-type mounting holes for the hand grip 3, the flow meter 13 and the heater control circuit 16. The case lid and case base when fastened together comprise the housing 2 which may be made out of a plastic to be lightweight and portable. The housing prevents the patient or other subject from touching any of the components and electronics contained therein.

The hand grip 3 is a handle secured to the housing via the opening and screw-type mounting holes, as described. The handle is held by the patient during measurements and supports the sensor rig 8 contained within the housing and comprising the components therein. The hand grip is hollow through which cables 9a,b,c are routed for convenience. The cables are connected to the computer and to a power source. Two USB cables 9a,b are for electronic communication and power delivery between the operating computer 20 and the CO₂ meter 10 and the flow meter 13. A third cable 9c carries power from an external supply or battery to the heater control circuit 16.

The mouthpiece 4 is disposable and may be a spirometry mouthpiece. The disposable mouthpiece is placed in the mouth of the patient or other subject who breathes through the mouthpiece for the duration of the measurement. It is discarded between patients. The mouthpiece is fluidly connected to the first filter or breath particle filter 5 which in combination forms a sub-assembly 7. The breath particle filter is disposable and is discarded between patients. The disposable breath particle filter keeps particles in the patient's breath from contaminating the device or damaging the CO₂ sensors contained within the CO₂ meter 10. A second filter or second breath particle filter 6 is a disposable intake filter that may be optionally used in dusty environments. The second filter is the same type as the first filter and is attached to the tubing connection on the flow meter 13.

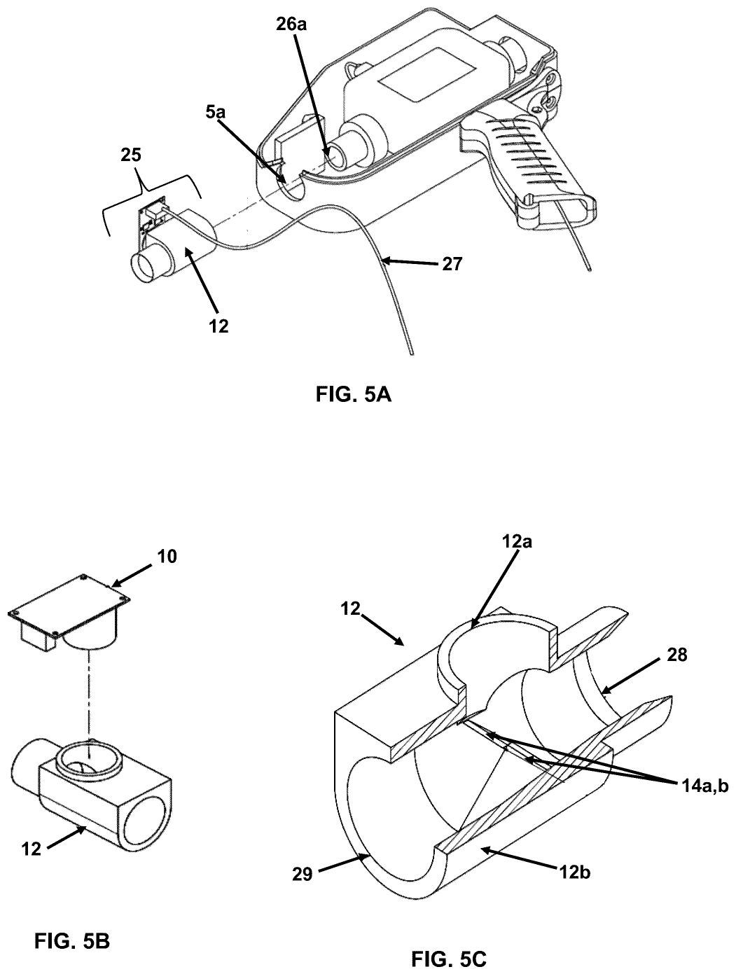

A CO₂ meter 10 is a component of the sensor rig 8 and is mounted up-side down within the flow chamber 12 (see FIG. 5B). The CO₂ meter uses at least one non-dispersive infrared (NDIR) CO₂ sensor that measures how much light from a 4.25 micron laser is absorbed by the gas from the breath passing through the meter to determine the relative concentration of CO₂ in that gas. The CO₂ meter reports these measurements electronically via the USB cables 9a to the operating computer. The operating computer sends the sensor power and commands to the CO₂ meter. The CO₂ meter is inserted into the interior of the flow chamber 12.

The flow chamber 12 is a specially constructed gas distributor which connects the CO₂ meter 10 and the flow meter 13 to patient breath collected from the mouthpiece 4. This gas distributor is heated to prevent condensation on interior surfaces and has a set of opposing baffles 14a,b (see FIG. 5C) which increase measurement speed and reliability through introduction of turbulence, and optimized flow geometry.

The flow meter 13 measures the volumetric flow rate of breath as it passes through the sensor rig 8 and has a display 13a to receive data viewable by the subject through opening 2e. The flow meter reports these measurements electronically via the cable 9b to the operating computer 20. The operating computer sends the sensor power and commands to the flow meter via the same cables. The flow meter is attached to a tubing connection 30 (see FIG. 5C) on the flow chamber 12. The end of the flow meter farthest from the patient is left open to fresh air or connected to the disposable second breath particle filter 6.

A resistive heater band 15 is an electronic resistive heater treated with an adhesive and applied to the flat surface 12b (see FIG. 5C) of the flow chamber 12. The resistive heater is used to heat the internal surfaces above the normal human body temperature and is controlled by the heater control circuit 16.

A heater control circuit 16 is a circuit which controls delivery of power to a resistive heater band 15 to maintain a consistent temperature above human body temperature. The heater control uses a simple thermistor and comparator circuit and relay for power delivery. The heater control circuit is connected to the heated band by four small cables routed through the interior of the device. The heater is mounted to the case base 2b using screws. A cable 9c delivers power to the heater control circuit and for delivery to the heater.

The breath analyzer is operably linked to the operating computer 20. The operating computer may be a standard laptop computer, such as, but not limited to, a DELL INSPIRON 3593, product of Dell Inc.) which is used to operate a program or algorithm or software which controls the meters, calculates the cardiac output from meter readings, and stores the measurements for review.

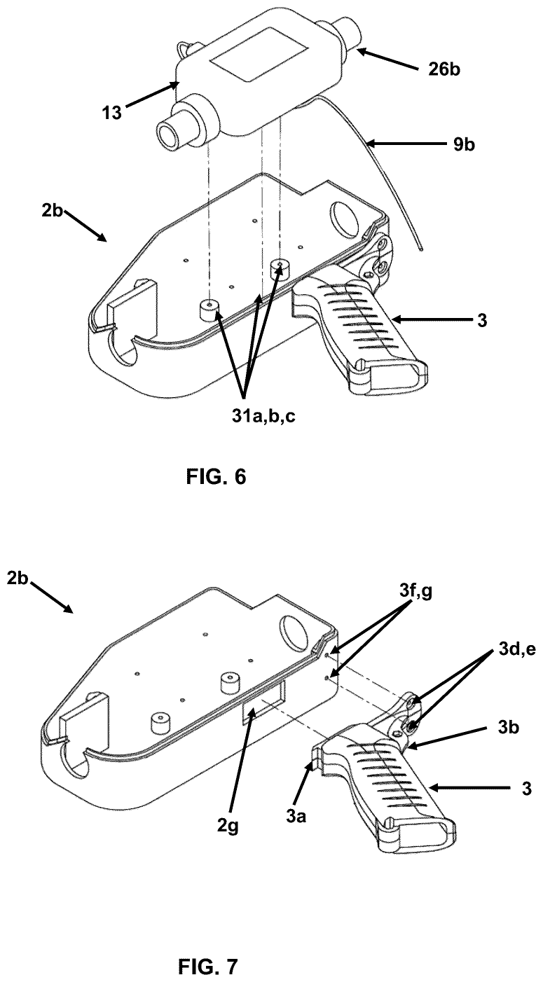

With continued reference to FIG. 1A and FIG. 2, FIGS. 3A-3C illustrate the assemblies of the mouthpiece-first filter combination and the second filter to the breath analyzer. FIG. 3A shows the assembly of the mouthpiece 4 and first filter 5 sub-assembly 7. The sub-assembly is made of disposable parts and is discarded between each patient. The sub-assembly is press fit to a 22 mm ID male tubing connector 28 (see FIG. 5C) on the flow chamber which is accessible through the inlet hole 5a in the housing 2. FIG. 3B shows the assembly of the components of the mouthpiece-filter sub-assembly 7. The disposable mouthpiece 4 is press-fit to the high-flow breath particle filter 5 at 18. FIG. 3C shows the attachment of the optional second filter 6 to the flow meter via a press fit 15 mm connection to the flow meter male outlet 26b (see FIG. 6) through outlet hole 6a in the housing 2. Both the first filter and the second filter are standard high-flow ventilator filters.

With continued reference to FIG. 2, FIG. 4 shows the assembly of the heater sub-assembly 21. The heater sub-assembly has the resistive heater band 15 with a Thorlabs HT10k resistive heater. The heated band is applied to the surface of the flow chamber 12 using an adhesive. Four cables, represented by 22, carry the heater power and thermistor voltage between the heater and heater control circuit which operates the heater. The heater control circuit comprises a circuit board 23. A power cable 24 connects the control circuit to a 12v DC power source.

With continued reference to FIG. 2, FIGS. 5A-5C shows the assembly of the flow chamber (FC)-CO₂ meter subassembly or CO₂ adapter tube. FIG. 5A shows that the FC-CO₂ subassembly 25 is attached by rocking the flow chamber 12 into the inlet hole in the housing at 5a, then sliding the flow chamber over the flow meter inlet male connector at 26a. A USB cable 27 carries sensor information from the CO₂ meter 10 to the operating computer 20. The operating computer sends power and commands to the device using this same cable. FIG. 5B is an exploded view of the FC-CO₂ subassembly. The CO₂ meter is a SPRINT-IR 6s CO₂ meter with a GSS NDIR gas sensing cavity. The USB cable has been excluded for clarity. The CO₂ meter is inserted upside down into the flow chamber at 12a perpendicular to the breath flow and rests on the opposing baffles 14a,b of the flow chamber. Because the breath flow comprises extremely humid air, the upside down position ensures that water droplets are pulled away from the CO₂ meter by gravity. FIG. 5C is a cross-section of the flow chamber showing the opposing baffles disposed within. Two baffles introduce turbulence into breath flow for faster measurements. These baffles are thin and offer low resistance to breath. Also shown are the male 28 and female 29 22 mm ID tubing connections for connection to the first filter 5 and to the flow meter 12, respectively, and the surface 12b for mounting the resistive heater band.

With continued reference to FIG. 2, FIG. 6 shows the assembly of the flow meter in the housing. The flow meter 13 is a TSI 5300 flow meter and is fastened to the case base 2b using three 3 mm screws 31a,b,c. The flow meter outlet male connector 26b is inserted into the outlet hole 6a on the case base, then rocked into place. A USB cable 9b carries sensor information to the operating computer 20. The operating computer sends power and commands to the device using this same cable. When assembled the USB cable extends into and through the hollow hand grip 3 to the operating computer.

With continued reference to FIG. 2, FIG. 7 shows how the case handle or hand grip 3 is attached to the case base 2b. The hollow hand grip has a first flange 3a and a second flange 3b extending outwardly from the opening at the top of the hand grip and substantially perpendicularly to the hand grip. The second flange has screw holes 3d,e. An opening 2g is formed in the case base such that the top opening in the hand grip aligns with the opening in the case base when the first flange hooks onto the edge thereof. The screw holes are threadably aligned to fasten the hand grip to the case base at 3f,g. This enables cables 9a,b,c to extend through the hand grip to the operating computer and to the heater control circuit.

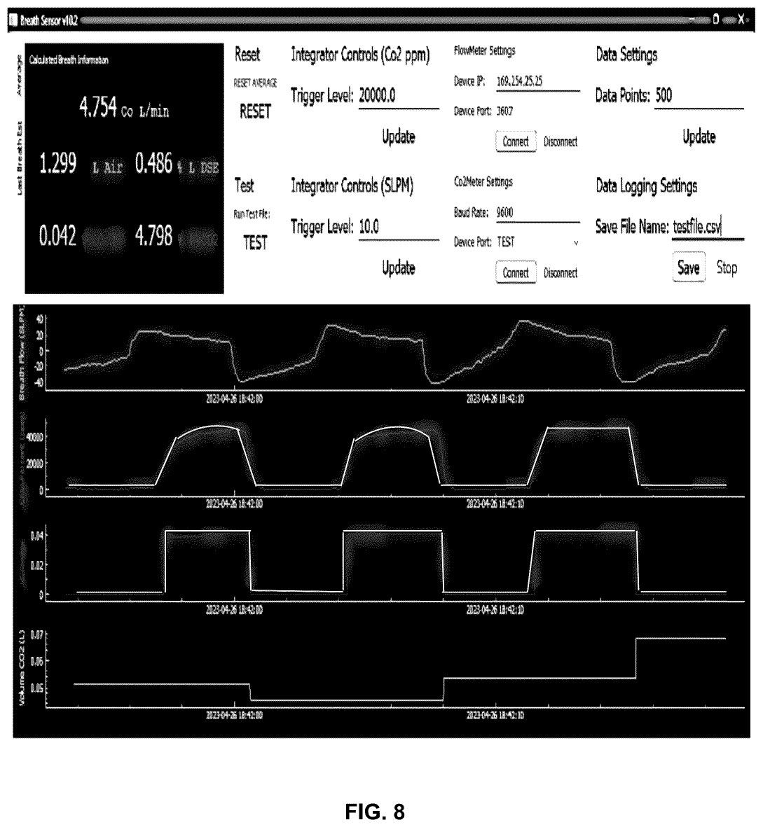

FIG. 8 is a screenshot showing the universal interface (UI) for the operating program.

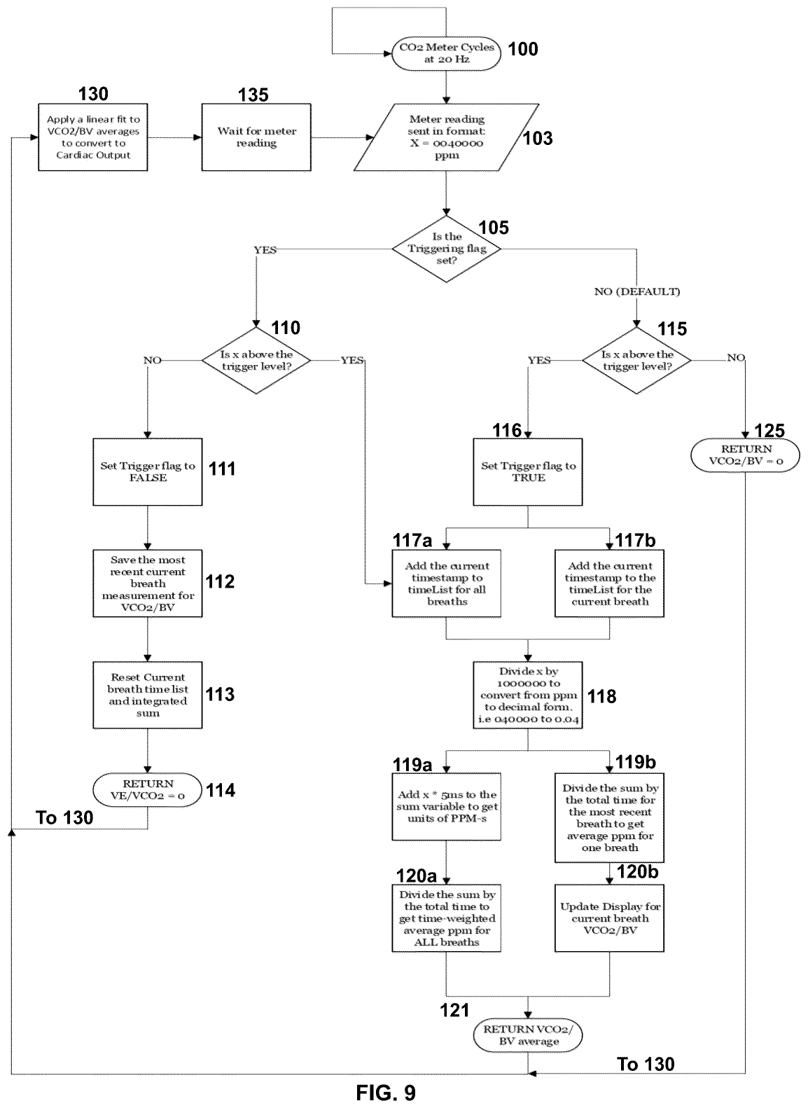

FIG. 9 is a flowchart of the computer analysis of exhalatory breath detected by the CO₂ meter. During breath measurement the CO₂ meter cycles at 20 Hz at 100 and the meter reading is sent in the format X=0040000 ppm at 103. The query Is the Triggering flag set? is made at 105. If the answer is YES, the query Is x above the trigger level? is made at 110. If the answer is NO, the Trigger flag is set to FALSE at 111, the most recent current breath measurement for VCO₂/BV is saved at 112, the current breath time list and the integrated sum is reset at 113, and VE/VCO2=0 is returned at 114. A linear fit is applied to VCO₂/BV averages to convert to cardiac output at 130 and the system waits for a meter reading at 135, then proceeds to 103.

If the query at 110 is YES, the algorithm proceeds to 117a which occurs when the answer to step 105 is NO or default and the answer to the query Is x above the trigger level? is YES at 115. In this instance, the Trigger flag is set to TRUE at 116. This is followed by step 117a during which the current timestamp is added to the time List for all breaths and the current timestamp is added to the timeList for the current breath at 117b. Then at step 118 the ppm are converted to decimal form by dividing by 1000000 to convert from 040000 to 0.04. At step 119a x*5 ms is added to the sum variable to units of PPMs followed by dividing the sum by the total time to get the time-weighted average ppm for ALL breaths at 120a. At step 119b the sum is divided by the total time for the most recent breath to get average ppm for one breath followed by updating the Display for current breath VCO₂/BV at 120b. Both 120a and 120b are followed by step 121 where the algorithm proceeds to RETURN VCO2/BV average. The algorithm then proceeds back to step 130. If the query at 115 is NO, the algorithm proceeds to step 125 to RETURN VCO₂/BV=0. The algorithm then proceeds back to step 130.

The following examples are given for the purpose of illustrating various embodiments of the invention and are not meant to limit the present invention in any fashion.

### Example 1

### Materials and Method

### Ambulatory Breath Analyzer

The device uses [CO₂] and volume of exhaled breath as input and has a heated breath flow path with baffles to account for the high humidity in breath. Exhaled flow rate of CO is measured, then divided by the difference between venous and arterial [CO₂]. The difference is calculated from exhaled CO₂ input to a formula derived in Example 2. Custom software then calculates cardiac output in real time. The exterior and interior components of the device are well-described in the figures.

### Computer

The operating software is built in Python. The computer runs an operating program or algorithm which delivers commands to and controls the CO₂ meter and the flow meter and collects data generated during cardiac output measurements. Collected data is quickly received as input and processed by the algorithm in real time and displayed to the user in a human-readable format (Live plots and regularly updating averages). Collected and calculated values also are organized and saved by the program. The operating program is configured to perform functions such as, but not limited to, the integration of flow readings into volume measurements, calculation of various metrics, i.e., VE, VE/VCO₂, % CO₂, display, and storage. The program may be operated on a standard laptop computer or a standard desktop computer or other smart or electronic device as are known in the art. The operating program uses a universal interface (UI) with intuitive plots and readouts for the operator. Latency is made as short as possible, so the operator is able to view real-time plots of readings and calculations.

### General Method

A subject or adult patient places the mouthpiece into his mouth and exhales into it. The breath is filtered to remove particles and enters the flow chamber where exhaled flow rate of CO₂ is measured, then divided by the difference between venous and arterial [CO₂]. The difference is calculated from exhaled CO₂ input by the novel CO₂-Modified Fick equation (Example 2). Custom software then calculates cardiac output in real time. Cardiac output is measured at rest and after a set pace treadmill jogging for healthy subjects. Cardiac output of patients with heart failure or other cardiac dysfunction is measured only at rest.

### Example 2

### Calculating Cardiac Output

### CO₂-Modified Fick Equation

The CO₂-Modified Fick equation is the Fick Equation for Cardiac Output using carbon dioxide instead of oxygen as the marker. Equations 1 and 2 are as follows:

$${Q}_{ECO 2} = ( {Q}_{CO} \times {C}_{VCO 2} ) - ( {Q}_{CO} \times {C}_{ACO 2} ) \qquad Eq . 1$$

$${Q}_{CO} = \frac{{Q}_{ECO 2}}{( {C}_{VCO 2} - {C}_{ACO 2} )} \qquad Eq . 2$$

Equation Legend

| Symbol | Units | Meaning |
|---|---|---|
| Q_{ECO2} | Liters/min | Flow rate of exhaled CO₂ |
| Q_{CO} | Liters/min | Flow rate of blood through the heart (cardiac |
| | | output) |
| Q_{VCO2} | Unitless (0-1) | Concentration of CO₂ in the mixed venous |
| | | blood |
| Q_{ACO2} | Unitless (0-1) | Concentration of CO₂ in the arterial blood |

### Input Values:

There are only two inputs required for this calculation: concentration of CO₂ in exhaled breath, and the flow rate of exhaled breath. Averages may be used, but real-time measurements are ideal.

The conduct of the measurement subject has a large effect on these measurements. Body position, respiration rate, exercise state, and many other small factors can modulate the cardiac output and strongly influence the quality of measurements. All measurements should be:
i. Stationary. Subjects should remain still in a sitting or prone position without moving during the measurement.
ii. Consistent. Subjects should breathe in a consistent and repeatable manner for measurements. Some preliminary testing indicated that a controlled rate of 4 seconds inhalation and 4 seconds exhalation were optimal.
iii. Repeated. Subjects should take many breaths in one measurement. 10 breaths is the typical number, but anything from 5-20 is adequate.
iv. Exclude the first measurement. Notably, the first breath in nearly every measurement has an artificially high CO₂ and is normally discarded.

### Volumetric Flow Rate of Exhaled Breath

Equation 3 is as follows:

$${Q}_{ECO 2} = {V}_{E} \times {C}_{ECO 2} \qquad Eq . 3$$

Equation Legend

| Symbol | Units | Meaning |
|---|---|---|
| Q_{ECO2} | Liters/min | Flow rate of exhaled CO₂ |
| Q_{E} | Liters/min | Flow rate of exhaled breath |
| C_{ECO2} | Unitless (0-1) | Concentration of CO₂ in the exhaled breath |

The numerator of the CO₂ modified Fick Equation may be represented by equation 3 above. This quantity is measured directly. The product of the measured CO₂ concentration and flow rate of exhaled breath provides this measure. The units for this measurement are carried to the final result, as the denominator of the CO₂ modified Fick equation is unitless.

Converting the Exhaled Concentration of CO₂ to Partial Pressure of CO₂ in the Mixed Venous Blood

Equations 4 and 5 are as follows:

$${P}_{VCO 2} = {C}_{ECO 2} \times ( 760 {e}^{- \frac{mgh}{RT}} - {P}_{H 2 O} ) + 33 \qquad Eq . 4$$

$${P}_{H 2 O} = 7.50062 \times 0.61121 {e}^{( 18.678 - \frac{T}{234.5} ) \times ( \frac{T}{257.14 + T} )} \qquad Eq . 5$$

Equation Legend

| Symbol | Units | Meaning |
|---|---|---|
| P_{VCO2} | Torr | Partial pressure of CO₂ in mixed venous blood |
| C_{ECO2} | Unitless (0-1) | Concentration of CO₂ in exhaled breath |
| 760 | Torr | Conversion factor from atm to Torr at STP |
| m | Kg/mol | Molar mass of air |
| g | m/s² | Gravitational acceleration constant |
| h | m | Altitude of concentration measurement |
| R | (N*m)/(mol*k) | Universal gas constant |
| T | k | Standard atmospheric temperature at altitude of |
| | | concentration measurement |
| P_{H2O} | Torr | Partial pressure contribution of water vapor |
| 30 | Torr | Constant offset between exhaled partial |
| | | pressure of CO₂ and venous partial pressure |
| | | of CO₂ |

The denominator of the CO₂-modified Fick equation is derived exclusively from measurements of exhaled concentration of CO₂. It is more convenient to first convert from a measurement of CO₂ concentration to a measurement of partial pressure of CO₂ in the exhaled breath for the following steps, so this is performed first.

At atmospheric pressure, the partial pressure of CO₂ would be equal to the product of CO₂ concentration and the atmospheric pressure adjusted for elevation. The simple version of the barometric formula is used to correct this calculation for altitude differences from sea level. The pressure is approximated at measurement and in the alveoli to be local atmospheric pressure over the duration of the breath.

The barometric formula is for dry gas, but the error at low altitudes is very small. A much larger contribution to the partial pressure is the vapor pressure of the water in exhaled breath. The relative humidity of water in exhaled breath is normally near 100 percent. The contribution of vapor pressure in exhaled breath to the total partial pressure can be taken as the vapor pressure of water at body temperature. The Buck Equation (2) given in equation 5 is used to calculate this pressure calculation, resulting in a constant contribution of 47.1 Torr.

Lastly, the partial pressure of CO₂ in exhaled breath is related to the partial pressure of CO₂ in the mixed venous blood. Table 1 compares the average partial pressure of CO₂ in exhaled breath to the average partial pressure of CO₂ in the mixed venous blood sample by Sun (3) over the course of exercise in healthy volunteer subjects. Comparing our rest values to their values of exercise after a short warmup and 5 minutes of exercise, it is clear that there is a constant offset of approximately 33 Torr for both exercise states. This term is added in equation 4 to reach the partial pressure of CO₂ in venous blood, as measured from exhaled CO₂.

TABLE 1

| Exercise State | Source | Average | Std. Deviation |
|---|---|---|---|
| R | Sun | 46.8 | 2.6 |
| | Us | 13.4388 | 3.7124 |
| E | Sun | 50.7 | 2.8 |
| | Us | 17.4721 | 3.8285 |

### Calculating the Difference Between Partial Pressure of CO₂ in the Mixed Venous Blood and Arterial Blood

Equation 6 is as follows:

$$Δ {P}_{V - A CO 2} = 0.0034 \times {( 0.4158 \times ( {P}_{VCO 2} - 33 ) )}^{3} + ( 0.4158 \times ( {P}_{VCO 2} - 33 ) ) \qquad Eq . 6$$

Equation Legend

| Symbol | Units | Meaning |
|---|---|---|
| ΔP_{V-A CO2} | Torr | Difference between partial pressure of mixed |
| | | venous and arterial blood |
| P_{VCO2} | Torr | Partial pressure of CO2 in mixed venous |
| | | blood |
| 0.0034 | Unitless | 3rd order scaling constant |
| −33 | Unitless | X intercept scaling constant |
| 0.4158 | Unitless | 1st order scaling constant |

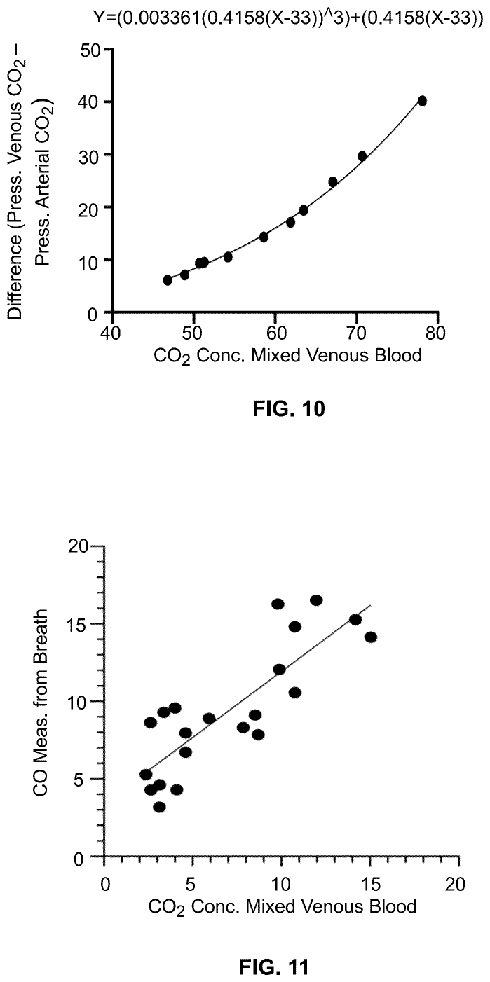

Equation 6 represents a critical step. This equation relates the difference in mixed venous and arterial blood partial pressures of CO₂ to the measured partial pressure of mixed venous CO₂. This equation was obtained by regression of the data presented in Sun et al. (3) and shown in FIG. 10A. The resulting regression curve is shown in FIG. 10B.

The construction of this equation for regression utilized the following logic:

The equation should be odd. Low CO₂ concentrations in the mixed venous blood should not result in an increase in CO₂ diffusion. This is especially true for values below the ‘zero point’ of approximately 33 Torr, which should be the minimum pressure necessary to diffuse CO₂ as implied by the offset of 33 Torr above.

The equation should be non-linear. A third power equation is the minimum which could describe this relationship while still adhering to the previous principle.

The inflection point should occur at the ‘zero point’ where CO₂ in the blood would be perfectly matched between the mixed-venous and arterial blood.

R for the resulting regression was 0.9980 with an R² of 0.9960, indicating a high quality of fit.

Converting the Partial Pressure of CO₂ in into Concentrations of CO₂ in the Blood

Equation 7 is as follows:

$${C}_{XCO 2} = \frac{462 \times {e}^{0.00415 \times {P}_{XCO 2}} - 340 \times {e}^{- 0.0445 \times {P}_{XCO 2}} + ( 97.5 - {S}_{XO 2} )}{1000} \qquad Eq . 7$$

Equation Legend

| Symbol | Units | Meaning |
|---|---|---|
| C_{ECO2} | Unitless (0-1) | Concentration of CO2 in X, where X is |
| | | either the mixed venous or arterial blood |
| 462 | Unitless | Scaling Constant from Regression |
| 0I00415 | Unitless | Scaling Constant from Regression |
| P_{ECO2} | Torr | Partial pressure of CO2 in x, where x is |
| | | either the mixed venous or arterial blood |
| 340 | Unitless | Scaling Constant from Regression |
| −0.0445 | Unitless | Scaling Constant from Regression |
| 07.5 | % | Scaling Constant from Regression |
| Sx_{O2} | % | Anticipated % CO2 saturation in x, where x |
| | | is either the mixed venous or arterial blood |
| 1000 | Unitless | Scaling Constant from Regression |

The denominator of the CO₂-Modified Fick Equation requires a difference of concentration instead of a difference in partial pressures. To facilitate this, the CO₂ disassociation curve was utilized. Equation 7 shows an empirical regression on the CO₂ Disassociation curve, fully detailed in Meade (4). This curve is convenient for algorithmic implementation. The arterial oxygenation saturation is estimated at 97.2%, and the venous oxygenation saturation is estimated at 71.1. %. These values are based on the O2 saturation values given by Sun et al. (3).

Concentrations of CO₂ can then be calculated in both the mixed-venous and arterial blood. The difference of these values then provides the denominator value necessary for final calculation of cardiac output.

### Example 3

Results with CO2-Modified Fick Equation

### Cardiac Output Correlation

These methods were tested on 11 healthy patients, both at rest and after a five minute treadmill exercise. Comparisons were made to cardiac output derived from echocardiogram measurements. The overall correlation was extremely good, with very low scaling error. There was some offset, as breath measurements consistently over-estimated both cardiac output and cardiac index by a small amount.

Cardiac output comparisons with echocardiogram measurements resulted in an excellent correlation of R=0.8344, R\hat{ }2=0.6962, and p<0.0001 from n=21 (FIG. 11). One measurement was excluded as an extreme outlier, >(75^{th} percentile)+1.5*SD. Linear regression showed low scaling error, as the slope of the comparisons was 0.8516 with a 95% confidence interval of [0.5815,1.122]. This, combined with the correlation R\hat{ }2 indicates a lack of non-linear error between these devices. There is a constant offset error present, indicated by the y intercept value of 3.410. This would be simple to correct for with calibration.

### Cardiac Output Bland-Altman

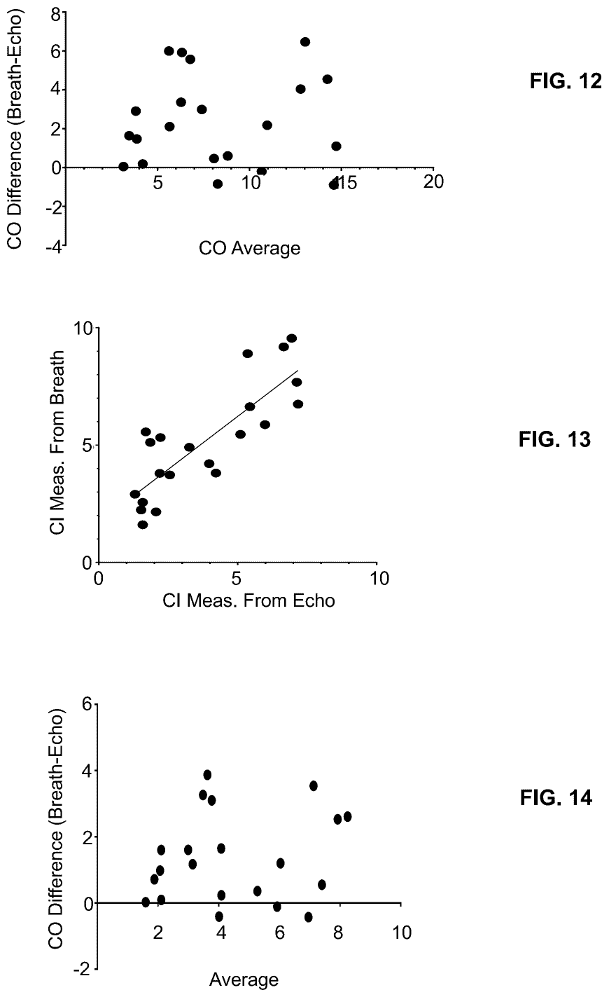

In FIG. 12 Bland-Altman analysis shows a bias of 2.364 with a 95% confidence interval of [−2.225,6.954]. There is, again, an indication of constant over-estimation of cardiac output by breath analysis. There do not appear to be any significant trends against the average measurement. Variance also appears constant across the measurement, providing further evidence that this fit is appropriate for the data.

### Cardiac Index Correlation

Cardiac Index measurements also show good correlation with echocardiogram measurements with r=0.8239, R\hat{ }2=0.6787, and p<0.0001 (FIG. 13). Linear regression shows a very low scaling error with slope of 0.9025 and 95% CI of [0.6044,1.201] indicating excellent agreement across all measured scales. There is little offset between the two devices, indicated by the low y intercept of 1.710 with 95% CI of [0.4171,3.003]. Cardiac Index was calculated as the cardiac output divided by the Mostellar BSA.

### Cardiac Index Bland-Altman

Bland-Altman analysis of cardiac index shows a bias of 1.340 with a 95% confidence interval of [−1.298,3.977] (FIG. 14). There is, again, an indication of constant over-estimation of CO by breath analysis. There do not appear to be any significant trends against the average measurement. Variance also appears constant across the measurement, providing further evidence that this fit is appropriate for the data.

### Example 4

### Results of Performance Testing of ABAMCO Device

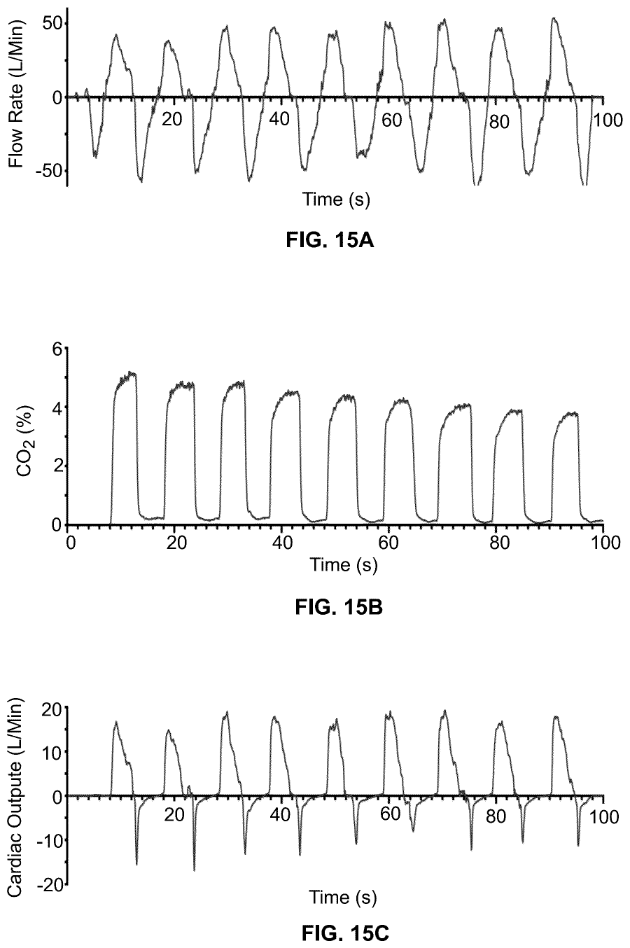

Eleven healthy volunteer subjects were recruited. Reference echocardiogram estimates of cardiac output obtained from stroke volume x heart rate were taken for each. Subjects were measured standing at rest, then performed a controlled exercise regimen. Subjects were measured immediately post exercise for 10 breaths. FIGS. 15A-15C show the results obtained over 100 sec. for flow rate, CO₂ concentration (%) and cardiac output.

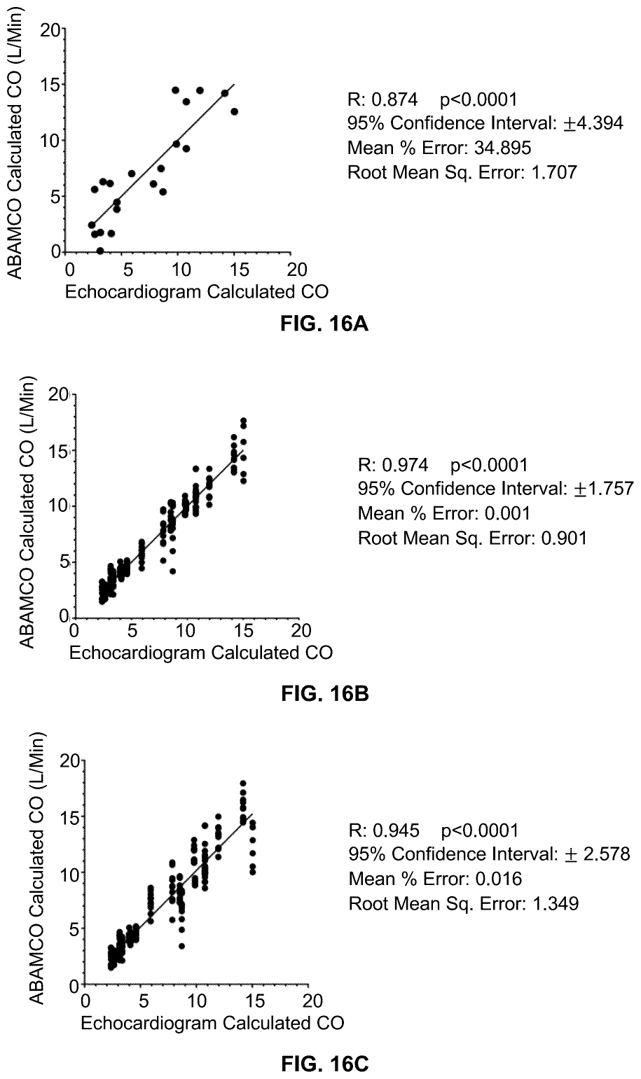

Calibration testing of the ABAMCO device was performed. Gain and offset parameters were used for the raw readings. One parameter was used to scale individual readings to the echocardiogram reference. Additional parameters for modeling the pulmonary arterial to pulmonary venous relationship were used. There are several calculation parameters that can be adjusted to population. ABAMCO calculated cardiac output vs. echocardiogram calculated cardiac output were plotted for uncalibrated ABAMCO, calibrated ABAMCO and rest calibrated ABAMCO (FIGS. 16A-16C). Low cardiac output measurements exhibit lower variability than high cardiac output measurements.

### REFERENCES

1. de Boode et al. Pediatric Research, 61:279-283, 2007.
2. Buck A L. Journal of Applied Meteorology and Climatology, 20:1527-1532, 1981.
3. Sun et al. Journal of Applied Physiology, 90:1798-1810, 2001.
4. Meade F. B J A: British Journal of Anaesthesia, 44:630-630, 1072.

What is claimed is:

1. A device for analyzing breath exhalations in a subject, comprising:
a housing;
a sensor rig disposed within the housing; and
a computer operably connected to the sensor rig and comprising an algorithm configured to collect and process data and to display results.

2. The device of claim 1, wherein the housing comprises:
a hand grip attached to a bottom surface of the housing; and
a mouthpiece and a first filter combination in fluid connection, said first filter in fluid connection with an inlet to the sensor rig.

3. The device of claim 2, wherein the housing further comprises a second filter in fluid connection with an outlet from the sensor rig.

4. The device of claim 2, wherein the mouthpiece is disposable.

5. The device of claim 1, wherein the sensor rig comprises:
a flow chamber in fluid communication with the mouthpiece and a first filter combination;
a CO₂ meter inserted within the flow chamber and in fluid communication therewith; and
a flow meter attached to the flow chamber in fluid communication with the CO₂ meter and configured to measure flow rate of breath through the flow chamber; said flow meter comprising a display configured to receive processed data.

6. The device of claim 5, wherein the flow chamber comprises:
a heated band disposed exteriorly on a surface of the flow chamber; and
a pair of baffles disposed within the flow chamber.

7. The device of claim 6, wherein the CO₂ meter is inserted upside down into and perpendicular to the flow chamber to rest on the set of baffles.

8. The device of claim 5, wherein the CO₂ meter comprises non-dispersive infrared sensors.

9. A method for measuring cardiac output in a subject, comprising:
exhaling a breath by the subject through a mouthpiece and first filter combination into the sensor rig in the device of claim 1; and
measuring a concentration of carbon dioxide in the breath and volume of breath exhaled by the subject via a CO₂ meter and a flow meter disposed within a flow chamber within the sensor rig.

10. The method of claim 9, further comprising repeating the method steps at least once to monitor levels of cardiac output in the subject.

11. The method of claim 9, wherein the measuring step comprises:
receiving the breath into the CO₂ meter disposed within a flow chamber in the sensor rig;
measuring a carbon dioxide concentration in the breath via the CO2 meter;
measuring the volume of the breath via the flow meter in fluid communication with the flow chamber;
inputting the measured carbon dioxide concentration and the volume of the exhaled breath into the algorithm;
processing the measured carbon dioxide concentration and volume of exhaled breath as cardiac output data; and
outputting the cardiac output data to a display.

12. The method of claim 11, further comprising heating the flow chamber to prevent formation of condensation from the breath exhaled therein.

13. The method of claim 11, wherein the receiving step comprises:
holding the device via a grip secured thereto;
self-inserting the mouthpiece into the mouth of the subject; and
exhaling the breath into the mouthpiece and first filter combination thereby flowing into the flow chamber.

14. The method of claim 7, wherein the subject is a healthy subject or a patient with a cardiac dysfunction.

15. A tool to monitor cardiac output in real-time in a patient in need thereof, comprising:
a portable, handheld breath analyzer, comprising:
a housing with a hand grip attached to an exterior bottom surface thereof and a mouthpiece and particle filter subassembly attached in fluid connection to an exterior front surface thereof;
a flow chamber-CO₂ meter subassembly disposed within the housing and in fluid connection with the particle filter and comprising opposing baffles disposed within the flow chamber and a resistive heater band disposed on an exterior surface thereof, said CO₂ meter inserted into the flow chamber to contact the opposing baffles;
a flow meter disposed in fluid connection with the CO₂ meter; and
a computer in electronic connection with the CO₂ meter and the flow meter, said computer configured to process measurements received as input data therefrom via an algorithm.

16. The tool of claim 15, further comprising another particle filter disposed on a back surface of the housing and in fluid connection with the flow meter.

17. The tool of claim 15, wherein the CO₂ meter is inserted upside down into and perpendicular to the flow chamber.

18. The device of claim 15, wherein the CO₂ meter comprises non-dispersive infrared sensors.

19. A method for monitoring cardiac output in real time in a patient in need thereof, comprising:
handholding the breath analyzer of claim 15 by the patient and exhaling a breath into the mouthpiece and particle filter subassembly, said breath flowing into the flow chamber-CO₂ meter subassembly;
measuring a carbon dioxide concentration in the breath as it flows through the CO₂ meter;
measuring the volume of the breath as it flows into the flow meter;
inputting the measured carbon dioxide concentration and the volume of the exhaled breath into the algorithm;
processing the measured carbon dioxide concentration and volume of exhaled breath as cardiac output data;
outputting the cardiac output data to a display; and
repeating the method steps at least once over a period of time, thereby monitoring the cardiac output of the patient.

20. The method of claim 19, wherein the patient has a cardiac dysfunction.
